(19) United States
(12) Patent Application Publication
(10) Pub. No.: US 2026/0137264 A1
(43) Pub. Date: May 21, 2026

(54) SEARCH SYSTEM AND METHOD FOR ENDOSCOPIC IMAGES

(71) Applicants: INVENTEC (PUDONG) TECHNOLOGY CORPORATION, Shanghai (CN); INVENTEC CORPORATION, Taipei City (TW)

(72) Inventors: Chun-Ti CHOU, Taipei City (TW); Wei-Chao CHEN, Taipei City (TW); Chih-Pin WEI, Taipei City (TW); Po Hsuan HUANG, Taipei City (TW); Po-Han HUANG, Taipei City (TW); Chiu-Jung LU, Taipei City (TW)

(73) Assignees: INVENTEC (PUDONG) TECHNOLOGY CORPORATION, Shanghai (CN); INVENTEC CORPORATION, Taipei City (TW)

(21) Appl. No.: 19/233,198

(22) Filed: Jun. 10, 2025

(30) Foreign Application Priority Data
Nov. 18, 2024 (CN) 202411651896.X

(51) Int. Cl. A61B 1/00; G06V 10/74; G06V 10/774

(52) U.S. Cl. CPC A61B 1/000094; A61B 1/000096; G06V 10/761; G06V 10/774; G06V 2201/031

(57) ABSTRACT

A search system and method for endoscopic images is proposed. In this method, a feature extraction model generates a target feature value, along with multiple first and second feature values based on a target image and multiple first and second source images from different areas of a human organ. Next, the similarity between each first and second feature value and the target feature value is calculated, followed by the computation of first and second reference values based on these similarities. If the first reference value is greater than the second, the source image with the highest similarity among the first values is selected as the search result; otherwise, the source image with the highest similarity among the second values is chosen as the result.

### CROSS-REFERENCE TO RELATED APPLICATIONS

This non-provisional application claims priority under 35 U.S.C. § 119(a) on Patent Application No(s). 202411651896.X filed in People Republic of China on Nov. 18, 2024, the entire contents of which are hereby incorporated by reference.

### BACKGROUND

### 1. Technical Field

This disclosure relates to an endoscopic imaging and image comparison, and provides a system and method for searching endoscopic images.

### 2. Related Art

Endoscopes are primarily used to examine the internal condition of the digestive tract to detect signs of ulcers, tumors, or other lesions. During an endoscopic examination, the physician captures and archives images upon discovering abnormalities, in order to document the examination result and provide a reference for subsequent diagnosis and treatment planning.

However, existing endoscopic equipment is unable to retrieve prior examination images in real time to compare during an examination. Comparing medical images taken at different times but from the same location is critical for clinical diagnosis and treatment decisions, as it may effectively assist the analysis of physician. Nonetheless, the characteristics of endoscopic images present significant challenges for comparison: on one hand, a single examination often generates dozens of images, which is far more than other medical imaging modalities such as X-rays, thereby increasing the workload of screening and comparison; on the other hand, endoscopic images do not display the overall anatomical outline of organs, making it difficult to determine the location and hard to directly identify corresponding position of two images.

As a result, image comparison, whether conducted during the examination or after the examination, imposes a burden on physicians. Currently, there is a lack of effective and efficient solutions, which not only reduces the efficiency of endoscopic examinations but may also lead to the oversight of critical pathological signs due to limitations in information flow.

### SUMMARY

In view of the foregoing, this disclosure provides a system and method for endoscopic image search that may effectively and efficiently compare endoscopic images, thereby providing physicians with real-time comparative information.

According to an embodiment of this disclosure, a search method for endoscopic images comprises a plurality of steps performed by a computing device, and the plurality of steps comprises: generating a target feature value, a plurality of first feature values and a plurality of second feature values by a feature extraction model based on a target image, a plurality of first source images and a plurality of second source images, respectively, the plurality of first source images and the plurality of second source images belonging to endoscopic images of different areas of a human organ, respectively, calculating a plurality of first similarities between the target feature value and the plurality of first feature values and calculating a plurality of second similarities between the target feature value and the plurality of second feature values, calculating a first reference value and a second reference value according to the plurality of first similarities and the plurality of second similarities, respectively, selecting a first source image corresponding to a highest similarity among the plurality of first similarities as a search result when the first reference value is greater than the second reference value, and selecting a second source image corresponding to a highest similarity among the plurality of second similarities as the search result when the first reference value is not greater than the second reference value.

According to an embodiment of this disclosure, a search system for endoscopic images comprises a storage device and a computing device. The storage device is configured to store a plurality of first source images and a plurality of second source images. The plurality of first source images and the plurality of second source images are endoscopic images of different areas of a human organ, respectively. The computing device is electrically connected to the storage device. The computing device is configured to generate a target feature value, a plurality of first feature values and a plurality of second feature values by a feature extraction model based on a target image, the plurality of first source images and the plurality of second source images, respectively, calculate a plurality of first similarities between the target feature value and the plurality of first feature values, calculate a plurality of second similarities between the target feature value and the plurality of second feature values, calculate a first reference value and a second reference value according to the plurality of first similarities and the plurality of second similarities, respectively, select a first source image corresponding to the highest similarity among the plurality of first similarities as a search result when the first reference value is greater than the second reference value, and select a second source image corresponding to the highest similarity among the plurality of second similarities as the search result when the first reference value is not greater than the second reference value.

In view of the above description, the search system and method for endoscopic images proposed by the present disclosure may extract an image feature by a deep learning model to extract feature values of a source image (e.g., an image recorded from a previous examination) and a target image (e.g., a real-time endoscopic image or the most recent examination record), respectively, and the feature values may be used as references for subsequent comparison processes. On the other hand, similarity between the target image and each source image may be calculated, and through calculating an overall similarity between the target image and a single region, thereby selecting the source image with the highest similarity from the region with the highest similarity.

### BRIEF DESCRIPTION OF THE DRAWINGS

The present disclosure will become more fully understood from the detailed description given hereinbelow and the accompanying drawings which are given by way of illustration only and thus are not limitative of the present disclosure and wherein:

FIG. 1 is a block diagram of a search system for endoscopic images according to an embodiment of the present disclosure;

FIG. 2 is a flowchart of a search method for endoscopic images according to an embodiment of the present disclosure; and

FIGS. 3 and 4 are flowcharts of two embodiments for training the feature extraction model.

### DETAILED DESCRIPTION

In the following detailed description, for purposes of explanation, numerous specific details are set forth in order to provide a thorough understanding of the disclosed embodiments. According to the description, claims and the drawings disclosed in the specification, one skilled in the art may easily understand the concepts and features of the present invention. The following embodiments further illustrate various aspects of the present invention, but are not meant to limit the scope of the present invention.

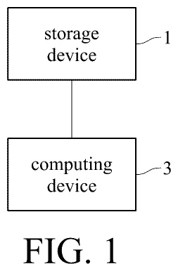

FIG. 1 is a block diagram of a search system for endoscopic images according to an embodiment of the present disclosure. As shown in FIG. 1, the system includes a storage device 1 and a computing device 3.

The storage device 1 is configured to store a plurality of first source images, a plurality of second source images, and a synthetic image or a composite image generated based on original images. The plurality of first source images and the plurality of second source images belong to endoscopic images of different regions of a human organ, respectively. For example, the plurality of first source images may be endoscopic images captured from the upper section of the intestine multiple times, and the second source images may be endoscopic images captured from the lower section of the intestine multiple times. Specifically, the storage device 1 stores all endoscopic images previously captured, and each image has one manually given image label to indicate which region of a human organ the image belongs. In other words, for a same organ, the endoscopic images stored in the storage device 1 may be categorized into a plurality of images of region 1, a plurality of images of region 2, . . . , and a plurality of images of region N. For illustrative purposes, the following descriptions refer to two regions, but the present disclosure does not limit on the no upper number of regions (N).

The storage device 1 may be a hard drive, a memory in a computer, or an external storage apparatus connected to a computer. In an embodiment, the storage device 1 may be implemented using at least one of the following examples: a flash memory, a hard disk drive (HDD), a solid state disk (SSD), a dynamic random-access memory (DRAM), a static random-access memory (SRAM), or other non-volatile memory. However, the disclosure is not limited to the above examples.

As shown in FIG. 1, the computing device 3 is electrically connected to the storage device 1. The computing device 3 is configured to train and execute a feature extraction model, calculate the overall similarity between a target image and different regions, calculate individual similarity between the target image and each source image, and select the region and/or source image with the highest similarity as the search result.

Specifically, the feature extraction model executed by the computing device 3 generates a target feature value, a plurality of first feature values and a plurality of second feature values based on a target image, a plurality of first source images and a plurality of second source images, respectively. The computing device 3 calculates a plurality of first similarities between the target feature value and the plurality of first feature values, and calculates a plurality of second similarities between the target feature value and the plurality of second feature values. The computing device 3 calculates a first reference value and a second reference value according to the plurality of first similarities and the plurality of second similarities, respectively. When the first reference value is greater than the second reference value, the computing device 3 selects the first source image corresponding to the highest similarity among the plurality of first similarities as a search result. When the first reference value is not greater than the second reference value, the computing device 3 selects the second source image corresponding to the highest similarity among the plurality of second similarities as the search result. It should be noted that the feature value described above may be represented in the form of a high-dimensional vector, a matrix, a tensor, or the like. In other words, the feature extraction model transforms images into high-dimensional data form, and the present disclosure is not limited thereto.

In an embodiment, the computing device 3 may use at least one of the following examples: a personal computer, a network server, a central processing unit (CPU), a graphics processing unit (GPU), a microcontroller unit (MCU), an application processor (AP), a field-programmable gate array (FPGA), an application-specific integrated circuit (ASIC), system-on-a-chip (SoC), a deep learning accelerator, or any other electronic device with similar functionality. The present disclosure does not limit the hardware type of the computing device 3.

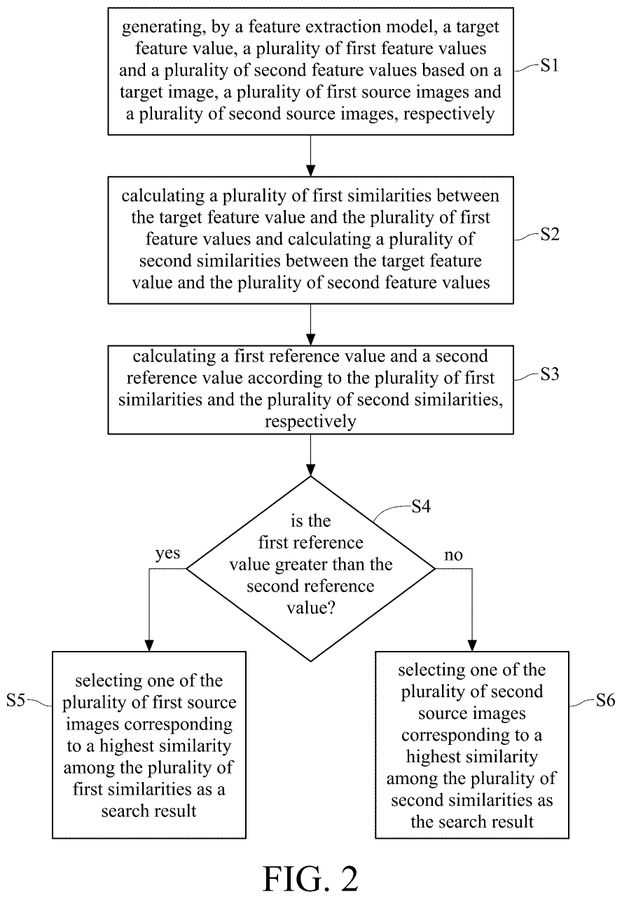

FIG. 2 is a flowchart of a search method for endoscopic images according to an embodiment of the present disclosure, including steps S1 to S5 executed by the computing device 3.

In step S1, the computing device 3 executes a feature extraction model, and the feature extraction model generates a target feature value, a plurality of first feature values, and a plurality of second feature values based on a target image, a plurality of first source images and a plurality of second source images, respectively.

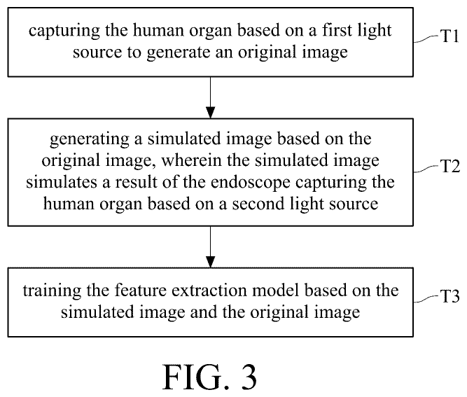

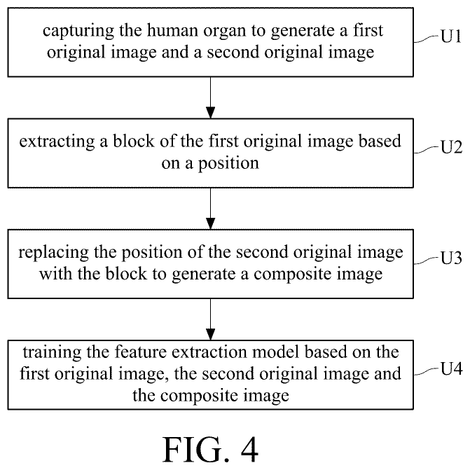

In an embodiment, the feature extraction model is trained using a self-supervised learning approach, and contrastive learning is used as a basic framework. The present disclosure proposes various image conversion methods that modify the original image while preserving the semantic content to generate multiple new images. In addition to common methods such as rotation and cropping, the disclosure provides two specific embodiments based on the features of endoscopic images. FIGS. 3 and 4 are flowcharts of two embodiments for training the feature extraction model.

The embodiment of FIG. 3 includes steps T1 to T3. In step T1, the endoscope captures the human organ based on a first light source to generate an original image. In step T2, the computing device 3 generates a simulated image according to the original image. The simulated image simulates the result of the endoscope capturing the human organ based on a second light source. In step T3, the computing device 3 trains the feature extraction model according to the simulated image and the original image. Specifically, in an embodiment, the first light source may be white light, and the second light source may be narrow band imaging (NBI), but the disclosure is not limited thereto. During an endoscopic examination, a physician may often switch between these two light sources. Therefore, the original image may be captured based on one of the two light sources. To enable the feature extraction model to extract features from the images of the same location under different light sources, the flow illustrated in FIG. 3 is adopted. By using the image conversion method of two light sources, the imaging effect of the same image under the other light source is simulated. In an embodiment, the computing device 3 modifies the specific color value in the original image, for example, reducing red color value or increasing green color value to simulate a narrow band image. The specific adjustment parameter may depend on the imaging differences of the first light source and second light source.

The embodiment illustrated in FIG. 4 includes steps U1 to U4. In step U1, the endoscope captures the human organ to generate a first original image and a second original image, wherein the first original image may be an endoscope image with an abnormal region, and the second original image may be an endoscope image without an abnormal region. The abnormal region in the original image may be manually labeled, or automatically labeled using an existing image recognition algorithm and a model. In step U2, the computing device 3 extracts a block of the first original image based on a position. The position is associated with the abnormal region of the captured human organ, such as inflammation, intestinal metaplasia, etc. In step U3, the computing device 3 replaces any position of the second original image with the block to generate a composite image, wherein the position of the second original image belongs to a normal region. Therefore, when preparing the dataset, all abnormal regions of an image need to be extracted in advance. During the training phase, each original image would obtain a random number. When the random number is greater than a predetermined threshold, an abnormal region image is randomly selected from the aforementioned abnormal region image library, and covers any one position of this original image. In step U4, the computing device 3 trains the feature extraction model based on the first original image, the second original image and the composite image.

By employing the two image conversion methods illustrated in FIGS. 3 and 4, the data volume for the computing device 3 when training the feature extraction model may be increased. The deep learning model may learn that feature values between different augmented images derived from the same image should be highly similar, while the feature values being significantly different from that of other images. Furthermore, for different parts of a same human organ, the embodiment of FIG. 3 or FIG. 4 may be applied multiple times so as to increase the data volume of each training dataset for different parts. Next, please turn back to the flowchart of FIG. 2.

In step S2, the computing device 3 calculates a plurality of first similarities between the target feature value and the plurality of first feature values, and calculates a plurality of second similarities between the target feature value and the plurality of second feature values In other words, the computing device 3 calculates the similarity between each source image and the target image based on the feature values. In an embodiment, the first similarities and the second similarities are Euclidean distance or cosine similarities.

In step S3, the computing device 3 calculates a first reference value and a second reference value according to the plurality of first similarities and the plurality of second similarities, respectively. In an embodiment, the first reference value is mean or median of the plurality of first similarities and the second reference value is mean or median of the plurality of second similarities, respectively. The first/second reference value is equivalent to the overall similarity between the target image and the region belonged to the first/second source image.

In step S4, the computing device 3 determines the relative magnitude of the first reference value with the second reference value. If the first reference value is greater than the second reference value, proceeding to step S5. If the first reference value is not greater than the second reference value, proceeding to step S6.

In step S5, when the first reference value is greater than the second reference value, the computing device 3 selects a first source image corresponding to the highest similarity among the plurality of first similarities as a search result.

In step S6, when the first reference value is not greater than the second reference value, the computing device 3 selects a second source image corresponding to the highest similarity among the plurality of second similarities as the search result.

The aforementioned process covers two stages such as comparing regional similarity (step S4) and comparing the similarity of each image (step S5 or step S6). The purpose of this design is to avoid highly similar images being scattered in different areas, resulting in the returned images not having regional consistency. In an embodiment, after identifying the region most similar to the target image, the computing device 3 selects the region with the highest similarity as the target of return, and returns an image similarity-ranked list of multiple images from that region, along with the similarity scores (reference values) of each region.

In view of the above description, the search system and method for endoscopic images proposed by the present disclosure may extract an image feature by a deep learning model to extract feature values of a source image (e.g., an image recorded from a previous examination) and a target image (e.g., a real-time endoscopic image or the most recent examination record), respectively, and the feature values may be used as references for subsequent comparison processes. On the other hand, similarity between the target image and each source image may be calculated, and through calculating an overall similarity between the target image and a single region, thereby selecting the source image with the highest similarity from the region with the highest similarity.

What is claimed is:

1. A search method for endoscopic images, comprising a plurality of steps performed by a computing device, and the plurality of steps comprising:
generating, by a feature extraction model, a target feature value, a plurality of first feature values and a plurality of second feature values based on a target image, a plurality of first source images and a plurality of second source images, respectively, wherein the plurality of first source images and the plurality of second source images are endoscopic images of different areas of a human organ, respectively;
calculating a plurality of first similarities between the target feature value and the plurality of first feature values and calculating a plurality of second similarities between the target feature value and the plurality of second feature values;
calculating a first reference value and a second reference value according to the plurality of first similarities and the plurality of second similarities, respectively;
selecting one of the plurality of first source images corresponding to a highest similarity among the plurality of first similarities as a search result when the first reference value is greater than the second reference value; and
selecting one of the plurality of second source images corresponding to a highest similarity among the plurality of second similarities as the search result when the first reference value is not greater than the second reference value.

2. The search method for endoscopic images according to claim 1, further comprising:
capturing, by an endoscope, the human organ based on a first light source to generate an original image;
generating, by the computing device, a simulated image based on the original image, wherein the simulated image simulates a result of the endoscope capturing the human organ based on a second light source; and
training, by the computing device, the feature extraction model based on the simulated image and the original image.

3. The search method for endoscopic images according to claim 1, further comprising:
capturing, by the endoscope, the human organ to generate a first original image and a second original image;
extracting, by the computing device, a block of the first original image based on a position;
replacing the position of the second original image with the block to generate a composite image; and
training, by the computing device, the feature extraction model based on the first original image, the second original image and the composite image.

4. The search method for endoscopic images according to claim 1, wherein the first reference value is mean or median of the plurality of first similarities and the second reference value is mean or median of the plurality of second similarities, respectively.

5. The search method for endoscopic images according to claim 1, wherein the plurality of first similarities and the plurality of second similarities are Euclidean distance or cosine similarities.

6. A search system for endoscopic images, comprising:
a storage device configured to store a plurality of first source images and a plurality of second source images, wherein the plurality of first source images and the plurality of second source images are endoscopic images of different areas of a human organ, respectively; and
a computing device electrically connected to the storage device, and configured to generate a target feature value, a plurality of first feature values and a plurality of second feature values by a feature extraction model based on a target image, the plurality of first source images and the plurality of second source images, respectively, calculate a plurality of first similarities between the target feature value and the plurality of first feature values, calculate a plurality of second similarities between the target feature value and the plurality of second feature values, calculate a first reference value and a second reference value according to the plurality of first similarities and the plurality of second similarities, respectively, select one of the plurality of first source images corresponding to the highest similarity among the plurality of first similarities as a search result when the first reference value is greater than the second reference value, and select one of the plurality of second source images corresponding to the highest similarity among the plurality of second similarities as the search result when the first reference value is not greater than the second reference value.

7. The search system for endoscopic images according to claim 6, wherein the storage device is further configured to store an original image generated by an endoscope capturing the human organ based on a first light source, and the computing device is further configured to generate a simulated image based on the original image and train the feature extraction model based on the simulated image and the original image, wherein the simulated image simulates a result of the endoscope capturing the human organ based on a second light source.

8. The search system for endoscopic images according to claim 6, wherein the storage device is further configured to store a first original image and a second original image generated by the endoscope capturing the human organ, and the computing device is further configured to extract a block of the first original image based on a position, replace the position of the second original image with the block to generate a composite image, and train the feature extraction model based on the first original image, the second original image and the composite image.

9. The search system for endoscopic images according to claim 6, wherein the first reference value is mean or median of the plurality of first similarities and the second reference value is mean or median of the plurality of second similarities, respectively.

10. The search system for endoscopic images according to claim 6, wherein the plurality of first similarities and the plurality of second similarities are Euclidean distance or cosine similarities.
